(19) United States
(12) Patent Application Publication
(10) Pub. No.: US 2013/0277450 A1
(43) Pub. Date: Oct. 24, 2013

(54) FRAGRANCE BOTTLE

(71) Applicant: ZHONGSHAN FUJI CHEMICAL CO., LTD., Zhongshan (CN)

(72) Inventor: Feifei FU, Zhongshan (CN)

(73) Assignee: ZHONGSHAN FUJI CHEMICAL CO., LTD., Zhongshan (CN)

(21) Appl. No.: 13/794,760

(22) Filed: Mar. 11, 2013

(30) Foreign Application Priority Data
Apr. 18, 2012 (CN) 201210115810.2

(51) Int. Cl. A61L 9/12

(52) U.S. Cl. CPC A61L 9/127
USPC 239/44; 239/34

(57) ABSTRACT

A bottle, including: a bottle body, the bottle body including a first opening and an inner cavity; a bottle cap, the bottle cap including a through hole; a bottle stopper, the bottle stopper including a plurality of holes. The bottle cap is disposed on the bottle body; the through hole is arranged on a top of the bottle cap; the bottle stopper is inserted into the first opening for sealing; and the holes are in communication with the inner cavity of the bottle body.

### CROSS-REFERENCE TO RELATED APPLICATIONS

Pursuant to 35 U.S.C. §119 and the Paris Convention Treaty, this application claims the benefit of Chinese Patent Application No. 201210115810.2 filed Apr. 18, 2012, the contents of which are incorporated herein by reference. Inquiries from the public to applicants or assignees concerning this document or the related applications should be directed to: Matthias Scholl P.C., Attn.: Dr. Matthias Scholl Esq., 14781 Memorial Drive, Suite 1319, Houston, Tex. 77079.

### BACKGROUND OF THE INVENTION

1. Field of the Invention

This invention relates to a bottle, and more particularly to a fragrance bottle.

2. Description of the Related Art

People like to use aroma oils to keep a fresh smell in their houses, and aroma twig diffusers are very popular in the market. A typical bottle for receiving aroma twig diffusers includes a bottle body and a bottle cap including holes; twigs are introduced through the holes of the cap, and directly inserted into the bottle body and immersed in the volatile matter. This structure is disadvantageous in that when the bottle is knocked down, the volatile matter in the bottle body quickly flows out of the bottle body, thereby resulting in waste.

### SUMMARY OF THE INVENTION

In view of the above-described problems, it is one objective of the invention to provide a fragrance bottle that has a simple structure, and can effectively prevent the inner fluids from leaking out of the bottle body when the bottle is knocked down.

To achieve the above objective, in accordance with one embodiment of the invention, there is provided a fragrance bottle comprises: a bottle body, the bottle body comprising a first opening and an inner cavity; a bottle cap, the bottle cap comprising a through hole; a bottle stopper, the bottle stopper comprising a plurality of holes. The bottle cap is disposed on the bottle body. The through hole is arranged on a top of the bottle cap. The bottle stopper is inserted into the first opening for sealing. The holes are in communication with the inner cavity of the bottle body.

In a class of this embodiment, the bottle stopper comprises: an upper end, and a connecting part. The connecting part is integrated with the upper end. The connecting part is plugged into the first opening of the bottle body.

In a class of this embodiment, the connecting part is in a shape of a conical cylinder. The connecting part comprises an outer wall; and an annular convex is disposed on the outer wall of the connecting part.

In a class of this embodiment, the bottle cap is in a shape of a cylinder. The bottle cap comprises: the top, and a bottom. A second opening is arranged on the bottom of the bottle cap. The second opening is sleeved on the bottle body. A diameter of the second opening is larger than a diameter of the top of the bottle cap.

In a class of this embodiment, the first opening of the bottle body is in a shape of a cylinder; and an external thread is arranged on an outer wall of the first opening.

In a class of this embodiment, an annular groove is arranged on a middle part of the bottle body. The second opening of the bottle cap comprises a convex clasp; the convex clasp is arranged on an inner wall of an end of the second opening; and the convex clasp matches with the annular groove.

In a class of this embodiment, a first recess is disposed on the top of the bottle cap; and the through hole is arranged on a bottom of the first recess.

In a class of this embodiment, the number of the holes arranged on the bottle stopper is seven; a diameter of each hole is 3.3 mm. One of the holes is arranged on a center of the upper end of the bottle stopper; and the other six holes are evenly distributed surrounding the center.

In a class of this embodiment, a twig is inserted into the through hole of the bottle cap; and a lower end of the twig is extended into the bottle body after passing through the holes arranged on the bottle stopper.

In a class of this embodiment, a second recess having a depth of 0.5 mm is arranged on the upper end of the bottle stopper. The connecting part comprises a cylinder cavity; and the cylinder cavity is in communication with the holes of the bottle stopper and the bottle body.

The bottle stopper is inserted into the first opening of the bottle body for sealing, and a plurality of holes are arranged on the bottle stopper. The size of the hole is comparable to the diameter of the twig. Thus, when the twigs are inserted into the holes of the bottle stopper, it assures that in condition that the bottle is knocked down, the inner fluid is prevented from leaking out of the bottle via the holes of the bottle stopper or other parts. The twig is extended to the volatile fluid after passing the through hole of the bottle cap and the holes of the bottle stopper. The bottle stopper comprises: the upper end, and the connecting part. The connecting part and the upper end are connected as a whole. The connecting part can be inserted into the first opening of the bottle body. The connecting part is a conical cylinder, which is very convenient to plug the bottle stopper into the first opening of the bottle body. A plurality of holes are arranged on the upper end of the bottle stopper. An annular convex is arranged on the outer wall of the connecting part. A diameter of the annular convex is a bit larger than a diameter of the inner wall of the first opening to assure a tight match between the connecting part and the first opening. The holes are evenly arranged on the upper end of the bottle stopper. A total of seven of the holes are arranged on the bottle stopper; the diameter of each hole is 3.3 mm. One of the holes is arranged on the center of the upper end of the bottle stopper; and the other six holes are evenly distributed surrounding the center. The second recess having the depth of 0.5 mm is arranged on the upper end of the bottle stopper. The cylinder cavity is in communication with the holes of the bottle stopper and the bottle body.

The fragrance bottle of the invention has a simple structure, and effectively prevents the volatile fluid from quickly leaking out of the fragrance bottle.

### BRIEF DESCRIPTION OF THE DRAWINGS

The invention is described hereinbelow with reference to the accompanying drawings, in which:

FIG. 1 is an assembly diagram of a fragrance bottle of the invention;

FIG. 2 is an exploded view of a fragrance bottle of the invention;

FIG. 3 is a stereograph of a bottle stopper; and

FIG. 4 is a cross-sectional view of a bottle stopper.

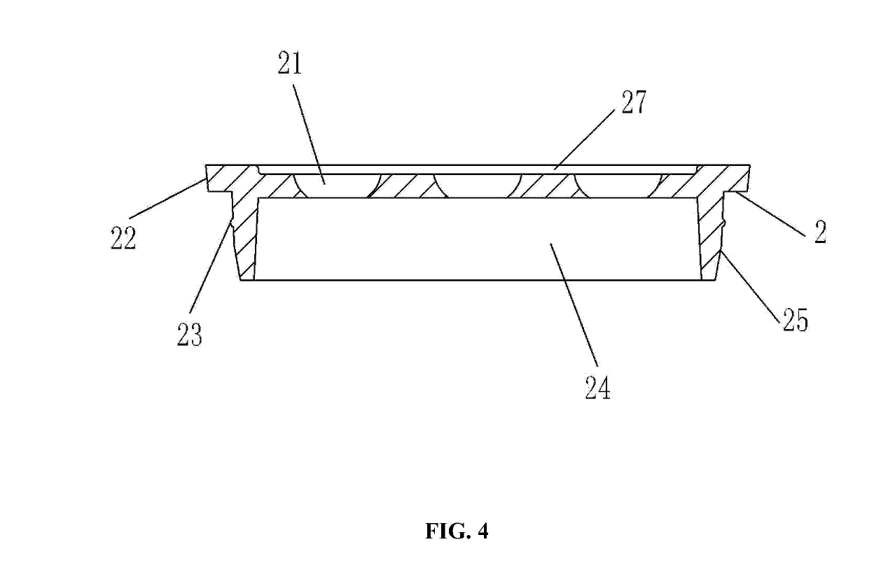

### DETAILED DESCRIPTION OF THE EMBODIMENTS

For further illustrating the invention, experiments detailing a fragrance bottle are described below. It should be noted that the following examples are intended to describe and not to limit the invention.

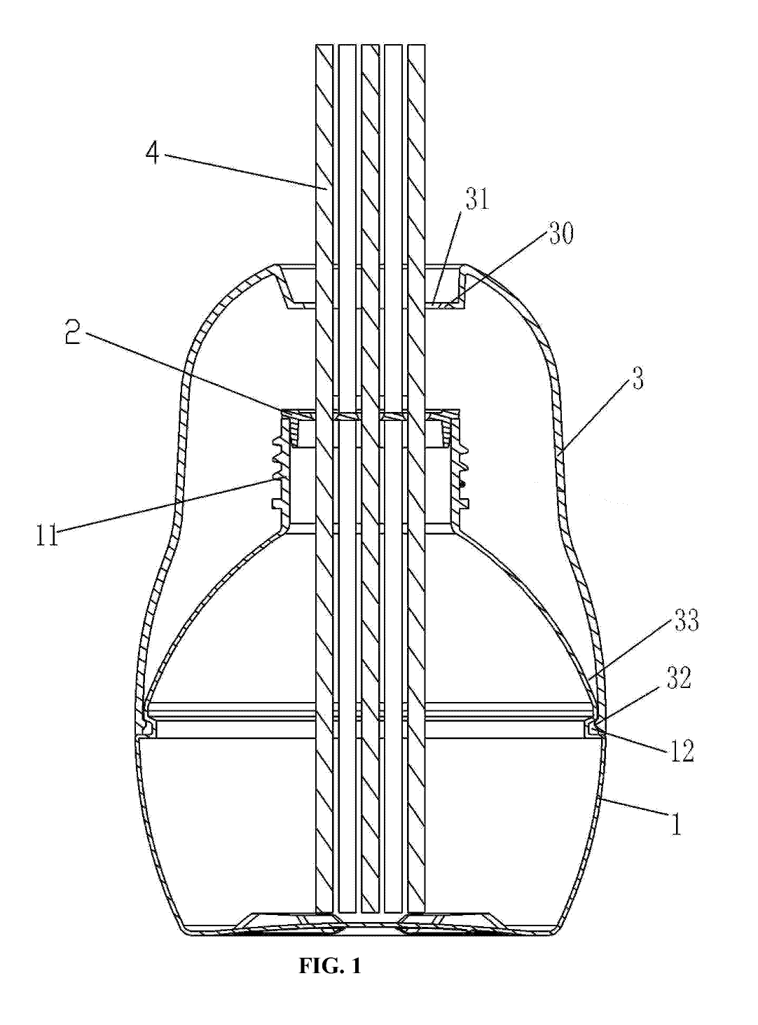

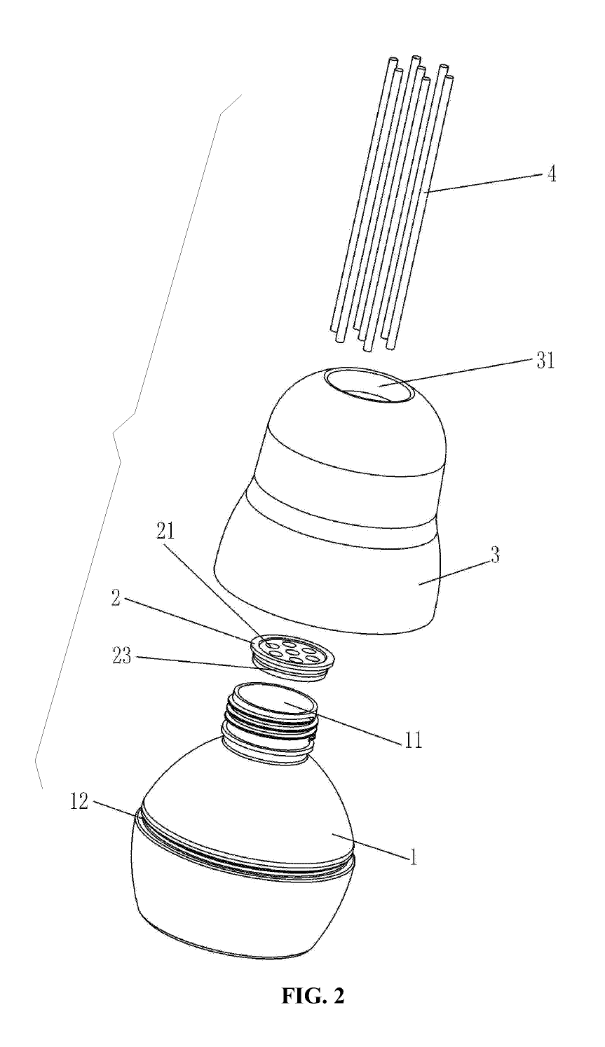

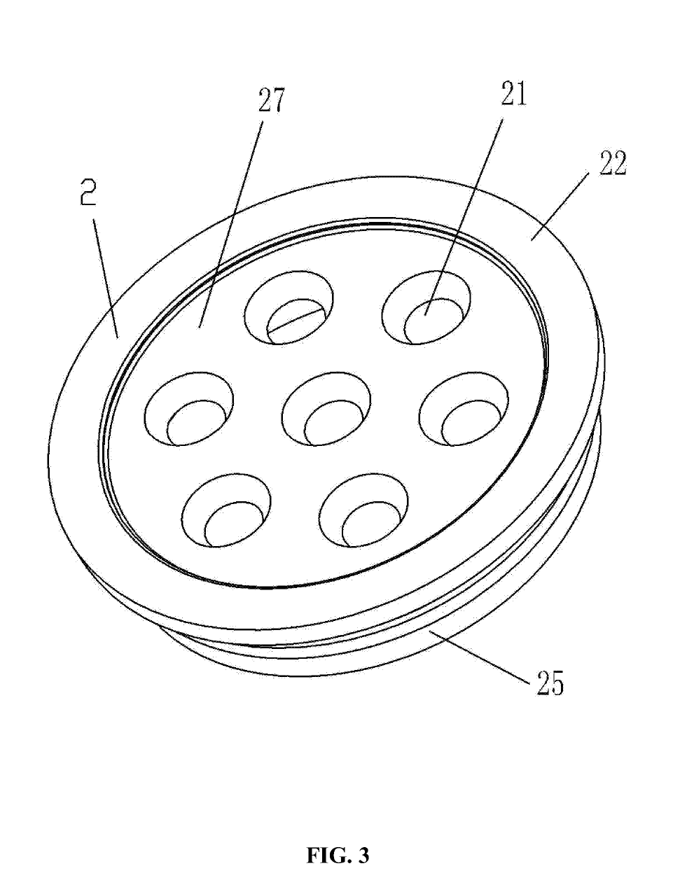

As shown in FIG. 1, a fragrance bottle comprises a bottle body 1 for filling an aroma fluid. The bottle body 1 comprises: an annular groove 12, an inner cavity, and a first opening 11. The annular groove 12 is arranged on a middle part of the bottle body 1. A bottle cap 3 is disposed on the first opening 11. The bottle cap 3 comprises: a top, and a bottom. A through hole 31 is arranged on the top, and a second opening 33 is arranged on the bottom, and the second opening 33 is sleeved on the bottle body. A diameter of the second opening 33 is larger than a diameter of the top of the bottle cap 3. A convex clasp 32 is arranged on an inner wall of an end of the second opening 33, and the convex clasp 32 matches the annular groove 12 of the bottle body 1. A bottle stopper 2 that tightly fits with the first opening 11 is inserted in the first opening 11. As shown in FIGS. 2 and 3, a plurality of holes 21 are arranged on the bottle stopper 2, the holes 21 are in communication with the inner cavity of the bottle body 1. A diameter of the hole 21 is comparable with a diameter of a twig 4, thus, when the twigs are inserted to the holes of the bottle stopper, it assures that in condition that the bottle is knocked down, the volatile fluid inside is prevented from leaking out of the bottle body 1 from the holes 21 of the bottle stopper or other parts. The twigs 4 are inserted into the bottle body 1 and immersed into the volatile fluid after passing the through hole 31 of the bottle cap 3 and the holes 21 arranged on the bottle stopper 2. The volatile fluid spreads along the twigs 4 from a lower end to an upper end, and spreads into the air via the holes 21 and the through hole 31.

A first recess 30 is arranged on the top of the bottle cap 3, and the through hole is arranged on a bottom of the first recess 30. Thus, when the twigs are inserted into the bottle body, the through hole is functions in supporting 4 in case of falling or bending of the twigs 4.

The bottle stopper 2 comprises: an upper end 22, and a connecting part 25. The connecting part 25 is integrated with the upper end 22; and the connecting part 25 is plugged into the first opening 11 of the bottle body 1. A plurality of the holes are arranged on the upper end of the bottle stopper 2. The connecting part 25 comprises an outer wall; and an annular convex is disposed on the outer wall of the connecting part 25. A diameter of the annular convex 23 is a bit larger than the diameter of the first opening 11 of the bottle body 1, which assures a good sealing property when the connecting part is inserted in the first opening 11. The connecting part 25 is in a shape of a conical cylinder, which is conducive to the sealing between the bottle stopper 2 and the first opening 11. The number of the holes 21 arranged on the bottle stopper 2 is seven. A diameter of each hole 21 is 3.3 mm. One of the holes 21 is arranged on a center of the upper end 22 of the bottle stopper 2; and the other six holes 21 are evenly distributed surrounding the center.

A second recess 27 having a depth of 0.5 mm is arranged on the upper end 22 of the bottle stopper 2. The connecting part 25 comprises a cylinder cavity 24; and the cylinder cavity 24 is in communication with the holes 21 of the bottle stopper 2 and the bottle body 1. In use, the twigs 4 are inserted into the through hole 31 of the bottle cap 3, and then introduced through the holes 21 of the bottle stopper 2, respectively. The connecting part 25 of the bottle stopper 2 is plugged into the first opening 11. The lower parts of the twigs are immersed into the volatile matters. Finally, the second opening 33 of the bottle cap 3 is sleeved on the bottle body 1, and the convex clasp 32 of the bottle cap 3 is received by the annular groove 12 on the bottle body 1.

The first opening 11 of the bottle body 1 is in a shape of a cylinder; and an external thread is arranged on an outer wall of the first opening 11. When a fragrance bottle is produced, a second bottle cap (not shown in FIGS.) is threaded on the first opening for sealing the bottle body. To use the product, at first the second bottle cap is removed from the first opening, then, the twigs are introduced through the through hole 31 of the bottle cap 3 and the holes 21 of the bottle stopper 31 until the lower parts of the twigs are immersed into the inner fluid.

While particular embodiments of the invention have been shown and described, it will be obvious to those skilled in the art that changes and modifications may be made without departing from the invention in its broader aspects, and therefore, the aim in the appended claims is to cover all such changes and modifications as fall within the true spirit and scope of the invention.

The invention claimed is:

1. A bottle, comprising:
a) a bottle body (1), the bottle body (1) comprising a first opening (11) and an inner cavity;
b) a bottle cap (3), the bottle cap (3) comprising a through hole (31);
c) a bottle stopper (2), the bottle stopper (2) comprising a plurality of holes (21);
wherein
the bottle cap (3) is disposed on the bottle body (1);
the through hole (31) is arranged on a top of the bottle cap (3);
the bottle stopper (2) is inserted into the first opening (11) for sealing; and
the holes (21) are in communication with the inner cavity of the bottle body (1).

2. The bottle of claim 1, wherein
the bottle stopper (2) comprises: an upper end (22), and a connecting part (25);
the connecting part (25) is integrated with the upper end (22); and
the connecting part (25) is plugged into the first opening (11) of the bottle body (1).

3. The bottle of claim 2, wherein
the connecting part (25) is in a shape of a conical cylinder;
the connecting part (25) comprises an outer wall; and
an annular convex is disposed on the outer wall of the connecting part (25).

4. The bottle of claim 1, wherein
the bottle cap (3) is in a shape of a cylinder;
the bottle cap (3) comprises: the top, and a bottom;
a second opening (33) is arranged on the bottom of the bottle cap (3);
the second opening (33) is sleeved on the bottle body (1); and
a diameter of the second opening (33) is larger than a diameter of the top of the bottle cap (3).

5. The bottle of claim 1, wherein
the first opening (11) of the bottle body (1) is in a shape of a cylinder; and
an external thread is arranged on an outer wall of the first opening (11).

6. The bottle of claim 1, wherein
an annular groove (12) is arranged on a middle part of the bottle body (1);
the second opening (33) of the bottle cap (3) comprises a convex clasp (32);
the convex clasp (32) is arranged on an inner wall of an end of the second opening (33); and
the convex clasp (32) matches with the annular groove (12).

7. The bottle of claim 5, wherein
an annular groove (12) is arranged on a middle part of the bottle body (1);
the second opening (33) of the bottle cap (3) comprises a convex clasp (32);
the convex clasp (32) is arranged on an inner wall of an end of the second opening (33); and
the convex clasp (32) matches with the annular groove (12).

8. The bottle of claim 1, wherein a first recess (30) is disposed on the top of the bottle cap (30); and the through hole (31) is arranged on a bottom of the first recess (30).

9. The bottle of claim 1, wherein
the number of the holes (21) arranged on the bottle stopper (2) is seven;
a diameter of each hole (21) is 3.3 mm;
one of the holes (21) is arranged on a center of the upper end (22) of the bottle stopper (2); and
the other six holes (21) are evenly distributed surrounding the center.

10. The bottle of claim 1, wherein a twig (4) is inserted into the through hole (31) of the bottle cap (3); and a lower end of the twig (4) is extended into the bottle body after passing through the holes (21) arranged on the bottle stopper (2).

11. The bottle of claim 2, wherein a second recess (27) having a depth of 0.5 mm is arranged on the upper end (22) of the bottle stopper (2); the connecting part (25) comprises a cylinder cavity (24); and the cylinder cavity (24) is in communication with the holes (21) of the bottle stopper (2) and the bottle body (1).
